(19) United States
(12) Patent Application Publication
(10) Pub. No.: US 2023/0381046 A1
(43) Pub. Date: Nov. 30, 2023

(54) CERVICAL TRACTION DEVICE

(71) Applicant: SHENZHEN AONUO MEDICAL TECHNOLOGY CO., LTD., Shenzhen, Guangdong (CN)

(72) Inventor: Zhenyu LI, Shenzhen, Guangdong (CN)

(21) Appl. No.: 18/031,948

(22) PCT Filed: Oct. 8, 2021
(86) PCT No.: PCT/CN2021/122684

§ 371 Date: Jun. 5, 2023

(30) Foreign Application Priority Data
Oct. 14, 2020 (CN) 202011099326.6

(51) Int. Cl. A61H 1/02

(52) U.S. Cl. CPC A61H 1/0218; A61H 1/0296; A61H 2201/10; A61H 2201/0207; A61H 2201/0221; A61N 2/002

(57) ABSTRACT

A cervical traction device, including: a base unit (3), a traction unit (1), a supporting unit (2), and at least one driving member (6). The traction unit (1) is rotatably mounted on the base unit (3); the supporting unit (2) is connected to the base unit (3), and the supporting unit (2) is arranged opposite the traction unit (1); and the driving member (6) is used for driving the traction unit (1) to rotate, such that the traction unit (1) is turned to or moves away from the supporting unit (2). The cervical traction device can allow a user to perform cervical traction by himself/herself, and is convenient to use.

### CROSS-REFERENCE TO RELATED APPLICATIONS

This application is the U.S. national phase of International Application No. PCT/CN2021/122684 with an international filing date of Oct. 8, 2021, designating the U.S., now pending, and claims the priority of the Chinese patent application number 202011099326.6 filed with the China Patent Office on Oct. 14, 2020 and titled “CERVICAL TRACTION DEVICE”, the entire contents each of which are incorporated in this application by reference.

### TECHNICAL FIELD

The present application relates to the technical field of medical devices, and more particularly to a cervical traction device.

### BACKGROUND

The statements herein merely provide background information related to the present application and do not necessarily constitute prior art.

In the era of intelligence, due to changes in life and work patterns, the incidence of cervical spondylosis has increased significantly, which accounts for nearly 35% of the population of spine clinics and seriously affects the normal life and work of patients. However, the current cervical traction is usually performed on a traction bed, which requires patients to go to the hospital for treatment, and it is inconvenient for patients to perform traction therapy at home, thereby bringing inconvenience to the patient's cervical spine treatment.

### SUMMARY

It is one of objectives of embodiments of the present application to provide a cervical traction device, which aims to solve the problem of how to make it easy for users to perform traction therapy by themselves.

In order to solve the above technical problems, the following technical solutions are adopted according to embodiments of the present application:

A cervical traction device is provided. The cervical traction device includes: a base unit, a traction unit, a support unit, and at least one driver member.

The traction unit is rotatably installed at the base unit, and the traction unit is configured to support a lower jaw of a user.

The support unit is in connection with the base unit and is arranged opposite to the traction unit; the support unit is configured to support a nape of the user.

The driver member is configured to drive the traction unit to rotate, to make the traction unit turn to or away from the support unit.

In an embodiment, the cervical traction device comprises two driver members, and the two driver members are disposed at two ends of the base unit, respectively.

The two driver members are respectively connected with two ends of the traction unit in a one-to-one correspondence, and the two driver members are configured to synchronously drive the traction unit to rotate.

In an embodiment, the traction unit is provided with a first connecting ring configured to connect with the base unit, and an outer wall of the first connecting ring is provided with a first limiting protrusion.

The base unit is provided with a corresponding second connecting ring, and the inner wall of the second connecting ring is provided with a second limiting protrusion.

The first connecting ring is inserted into the second connecting ring, and the second limiting protrusion is configured to limit a rotation stroke of the first limiting protrusion and in turn to limit a rotation stroke of the traction unit.

In an embodiment, at least one of the traction unit and the support unit is provided with a pressure detection assembly; and the pressure detection assembly is configured to detect a magnitude of a traction force that the traction unit exerts on the user.

In an embodiment, the pressure detection assembly comprises at least one pressure sensor.

In an embodiment, the support unit is provided thereon with a magnetic therapy sheet; and the magnetic therapy sheet is arranged at a side of the support unit that is close to a nape of the user.

In an embodiment, the support unit is further provided thereon with a heating sheet; and the heating sheet is arranged at a side of the support unit that is close to a nape of the user.

In an embodiment, a first flexible pad is arranged at a side of the traction unit close to the user, a second flexible pad is arranged at a side of the support unit close to the user, and a third flexible pad is arranged at a side of the base unit close to the user.

In an embodiment, the support unit is in connection with the base unit through a connection unit, and the support unit is installed at the connection unit. One end of the connection unit is in hinge connection with the base unit, and another end of the connection unit is in snap-fit connection with the base unit.

In an embodiment, the connection unit comprises: a first connecting belt, a second connecting belt, and an adjustment knob; the adjustment knob is rotatably installed at the support unit.

One end of the first connecting belt is in hinge connection with the base unit, and another end of the first connecting belt is inserted into the support unit and engaged with the adjustment knob.

One end of the second connecting belt is in snap-fit connection with the base unit, and another end of the second connecting belt is inserted into the support unit and engaged with the adjustment knob.

The adjustment knob is configured to drive the first connecting belt and the second connecting belt to move toward or away from each other by rotation, so as to lengthen or shorten a length of the connection unit.

In an embodiment, the traction unit further comprises a support member. The first flexible pad is arranged at a side of the support member close to the lower jaw of the human body. The first flexible pad matches a shape of a middle portion of the support member.

In an embodiment, the support unit further comprises a support component in connection with the base unit, and the second flexible pad is disposed at a side of the support component close to the traction unit.

In an embodiment, the support component comprises a first support plate and a second support plate, the first support plate and the second support plate are attached to each other, the first support plate is provided therein with a sliding chute and an installation groove, and the installation groove is in communication with the sliding chute.

In an embodiment, the base unit further comprises a housing assembly, and the third flexible pad is in connection with the housing assembly.

The beneficial effects of the cervical traction device provided by embodiments of the present application are summarized as follows:

In use, the cervical traction device can be directly placed around the user's neck, the base unit can be placed on the user's shoulder and chest; the support unit can be configured to support the user's nape, and the traction unit can be configured to support the user's lower jaw. When the driver member drives the traction unit to rotate toward the support unit, the user's lower jaw is driven to gradually rise; and when the driver member drives the traction unit to rotate away from the support unit, the user's lower jaw can be released. In this way, the traction of the user's cervical spine is realized, which enables the user to operate by themselves at home, and it is convenient for users to perform cervical traction therapy.

### BRIEF DESCRIPTION OF THE DRAWINGS

In order to more clearly illustrate the technical solutions in the embodiments of the present application, the drawings that need to be used in the description of the embodiments or the prior art will be briefly described hereinbelow. Obviously, the accompanying drawings in the following description are only some embodiments of the present application. For those skilled in the art, other drawings can be obtained based on these drawings without creative work.

FIG. 1 is a schematic structural diagram of a cervical traction device from a perspective;

FIG. 2 is a schematic structural diagram of a cervical traction device from another perspective;

FIG. 3 is a schematic diagram of an internal structure of a base unit;

FIG. 4 is a partial enlarged structural schematic diagram of Part A taken from FIG. 3;

FIG. 5 is a partial schematic structural diagram of a base unit;

FIG. 6 is a schematic structural diagram of a partial cross-section of a base unit;

FIG. 7 is an exploded schematic structural diagram of a traction unit;

FIG. 8 is a partial enlarged structural schematic diagram of Part B taken from FIG. 7;

FIG. 9 is a schematic structural diagram of a traction unit;

FIG. 10 is a partial enlarged structural schematic diagram of Part C taken from FIG. 9;

FIG. 11 is a schematic structural diagram of an assemblage of a support unit and a connection unit;

FIG. 12 is a schematic structural diagram of a support unit;

FIG. 13 is a schematic cross-sectional structure of a support unit;

FIG. 14 is a schematic structural diagram of a connection unit;

FIG. 15 is a schematic structural diagram of a first connecting belt;

FIG. 16 is a schematic structural diagram of a second connecting belt; and

FIG. 17 is a schematic structural diagram of a remote controller.

### DETAILED DESCRIPTION OF THE EMBODIMENTS

In order to make the purposes, technical solutions, and advantages of the present application clearer and more understandable, the present application will be further described in detail hereinafter with reference to the accompanying drawings and embodiments. It should be understood that the embodiments described herein are only intended to illustrate but not to limit the present application.

It should be noted that when an element is described as “fixed” or “arranged” on/at another element, it means that the element can be directly or indirectly fixed or arranged on/at another element. When an element is described as “connected” to/with another element, it means that the element can be directly or indirectly connected to/with another element. The orientation or positional relationship indicated by the terms “upper”, “lower”, “left”, “right”, and the like is based on the orientation or positional relationship shown in the drawings, and is only for the convenience of description, rather than indicating or implying the referred device or element must have a specific orientation, be constructed and operated in a specific orientation, and therefore should not be construed as a limitation on the present application, and those of ordinary skill in the art can understand the specific meanings of the above terms according to specific situations. Terms like “first” and “second” are only used for the purpose of description, and will in no way be interpreted as indication or hint of relative importance or implicitly indicate the number of the referred technical features. In the description of the present application, “multiple”/“a plurality of” refers to the number of two or more than two, unless otherwise clearly and specifically defined.

In order to illustrate the technical solutions described in the present application, a detailed description is given below with reference to the specific drawings and embodiments.

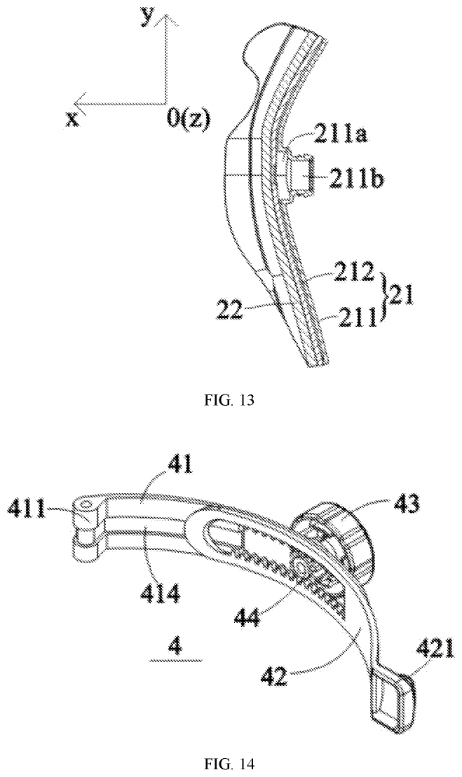

As shown in FIG. 13, a Cartesian coordinate system is established, in which, an x-axis direction and a y-axis direction are defined, and a direction perpendicular to a paper surface is defined as a z-axis direction.

### Example 1

This example provides a cervical traction device, which can perform traction therapy on a user's cervical vertebra. Meanwhile, the cervical traction device provided by the present application is convenient for users to perform cervical traction by themselves, and is convenient for users to use.

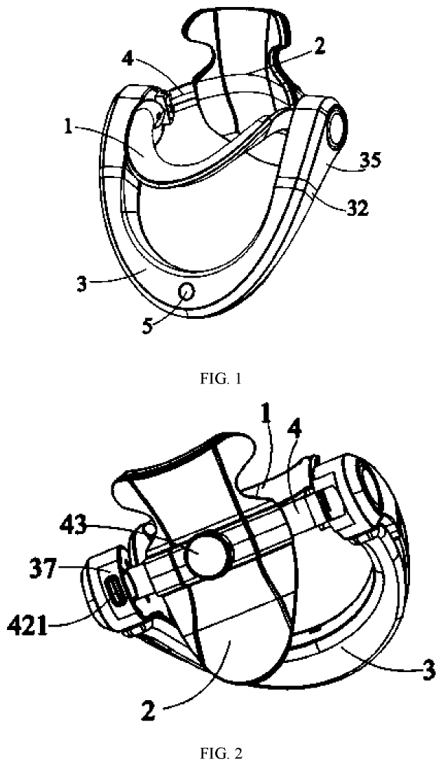

As shown in FIGS. 1, 2 and 7, the cervical traction device includes: a base unit 3, a traction unit 1, a support unit 2, and at least one driver member 6. The traction unit 1 is rotatably installed on the base unit 3; the support unit 2 is also in connection with the base unit 3, and the support unit 2 is arranged opposite to the traction unit 1. In use, the traction unit 1 is configured to support a user's lower jaw, and the support unit 2 is configured to support the user's nape.

The driver member 6 is configured for driving the traction unit 1 to rotate, so as to make the traction unit 1 turn to or move away from the support unit 2. Specifically, the driver member 6 is installed in the base unit 3 and is disposed near the connection between the traction unit 1 and the base unit 3. An output shaft 61 of the driver member 6 is connected with the traction unit 1, so that the traction unit 1 is driven by the driver member 6 to rotate.

In use, the user can arrange the cervical traction device around the neck, and place the base unit 3 on the shoulder and chest. The support unit 2 is disposed corresponding to the nape, to enable the support unit 2 to provide support for the nape. The traction unit 1 is disposed corresponding to the lower jaw of the user, to enable the traction unit 1 to provide support for the lower jaw portion. When the cervical traction device operates, the driver member 6 drives the traction unit 1 to turn to or move away from the support unit 2, thereby driving the user's lower jaw to be lifted or lowered. Specifically, when the driver member 6 drives the traction unit 1 to gradually rotate in a direction towards the support unit 2, the traction unit 1 gradually contacts the user's lower jaw and gradually lifts the user's lower jaw. When the driver member 6 drives the traction unit 1 to gradually rotate and reset in a direction away from the support unit 2, the traction unit 1 gradually releases the lower jaw of the user, so that the lower jaw can be lowered and reset. The reciprocating action of the cervical traction device can drive the user's lower jaw to be reciprocately lifted and lowered, so as to play a traction effect on the user's cervical vertebra.

The cervical traction device provided by the present application can be operated and used by the user, which is convenient for the user to perform cervical vertebra traction. Meanwhile, in the process of cervical traction, the user can adopt various postures, such as lying, sitting, standing, and the like, and is not limited to a specific posture, so the user experience can also be improved.

### Example 2

This example provides a cervical traction device. It can be understood that Example 2 makes further improvements based on Example 1.

As shown in FIG. 1, the base unit 3 has a U-shaped structure. The base unit 3 has a larger projected area than that of the traction unit 1, such that the base unit 3 can be placed more stably on the user's shoulders and chest to provide stable support. It can be understood that, in use, an open end of the U-shaped structure of the base unit 3 is disposed toward the user's neck, that is, the open end of the U-shaped structure is positioned at the user's shoulder, and the arc-shaped bottom of the U-shaped structure is positioned close to the user's chest.

Correspondingly, the side of the base unit 3 for contacting the user's shoulder has an arc-shaped structure, so as to fit the user's shoulder, so that the base unit 3 can be stably placed on the user's shoulder.

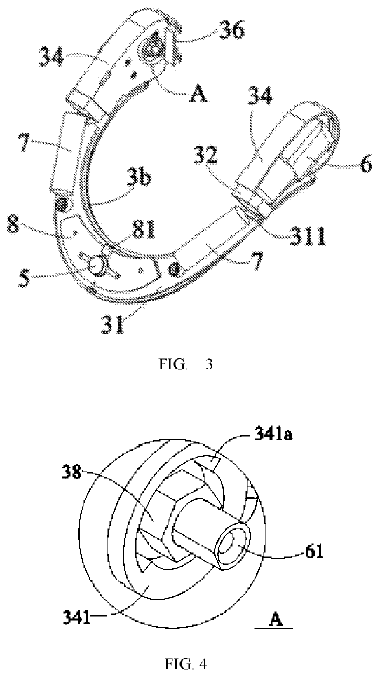

As shown in FIG. 1 and FIG. 3, the base unit 3 includes: a housing assembly 3a and a third flexible pad 3b. The housing assembly 3a has a U-shaped structure, thereby forming the basis for the U-shaped structure of the base unit 3. The third flexible pad 3b has a U-shaped structure adapted to the housing assembly 3a. The third flexible pad 3b is disposed on a side of the housing assembly 3a that is close to the user. That is, in the y-axis direction, the third flexible pad 3b is disposed beneath the housing assembly 3a. The third flexible pad 3b can be fixed on the housing assembly 3a by means of bonding, snapping, or the like.

In an embodiment, the third flexible pad 3b can be one selected from flexible pads made of, for example, a sponge, a silica gel, and a rubber. In an embodiment, the third flexible pad 3b is made of a sponge. It can be understood that a layer of cloth can be covered outside the sponge.

The housing assembly 3a includes: a bottom plate 31 and a first cover plate 33. The first cover plate 33 is covered on the bottom plate 31; and an accommodating chamber is formed between the first cover plate 33 and the bottom plate 31, and is configured for accommodating other components of the cervical traction device, such as the electronic devices that control the work of the cervical traction device. The fixed connection between the first cover plate 33 and the bottom plate 31 can be realized by means of a snap-fit connection, a screw connection, and the like. Both the bottom plate 31 and the first cover plate 33 have U-shaped structures, so as to form the basis for the U-shaped structure of the base unit 3.

As shown in FIG. 3, FIG. 5 and FIG. 6, the housing assembly 3a further includes two accommodating shells 34, the two accommodating shells 34 are respectively disposed on two ends of the bottom plate 31, and the two accommodating shells 34 are symmetrically arranged. In addition, the two accommodating shells 34 are in plug-in connection with the two ends of the first cover plate 33, respectively. The ends of the third flexible pad 3b extend to the contact positions between the accommodating shells 34 and the user, so as to ensure the use comfort degree of the user.

Optionally, each of both ends of the first cover plate 33 is provided with a first insertion portion 331; each of both housing shells 34 is provided with a second insertion portion 342 matched with the first insertion portion 331. When the accommodating shells 34 are in connection with the first cover plate 33, the first inserting portion 331 is in plug-in connection with the second inserting portion 342, and then the first inserting portion 331 and the second inserting portion 342 are locked and fixed together by screws. In this way, the fixed connection between the accommodating shells 34 and the first cover plate 33 is realized. It can be understood that the first cover plate 33 and the accommodating shells 34 are firstly connected by riveting, and then fixed by screws. Thus, the connection strength between the first cover plate 33 and the accommodating shells 34 can be enhanced, and the strength of the housing assembly 3a is enhanced as well.

A fixing ring 32 is also provided at a connection between the first inserting portion 331 and the second inserting portion 342, and the fixing ring 32 is sleeved outside the second inserting portion 342. On the one hand, the fixing ring 32 can fill a groove structure outside the second inserting portion 342, to allow a surface of the base unit 3 to be smooth; on the other hand, the fixing ring 32 can strengthen a strength of the second inserting portion 342, thereby ensuring the connection strength between the second inserting portion 342 and the first inserting portion 331.

A middle part of the first insertion part 331 is provided with a wire hole 331a; the second insertion part 342 is also provided with a wire hole communicated with the wire hole 331a, which enables a wire to pass through the connection between the first cover plate 33 and each of the accommodating shells 34.

As shown in FIG. 6, each of the two ends of the bottom plate 31 is provided with a buckle 311; and an inner part of the first insertion portion 331 of the first cover plate 33 is provided with a step matching with the buckle 311. In this way, when the first cover plate 33 is covered on the bottom plate 31, the buckle 311 can be fastened on the step inside the first insertion portion 331, so as to realize the pre-fixation of the first cover plate 33 and the bottom plate 31. In addition, the buckles 311 at two ends of the bottom plate 31 are in snap-fit connection with the steps arranged at two ends of the first cover plate 33, which also function in pre-positioning the first cover plate 33 and the bottom plate 31 and enables the first cover plate 33 and the bottom plate 31 to be locked and fixed via screws subsequently.

As shown in FIG. 1 and FIG. 3, each accommodating shell 34 has an open end so that other components can be installed in the accommodating shell 34. The open ends of the two accommodating shells 34 are disposed opposite to each other. In an embodiment, the housing assembly 3a further includes two second cover plates 35, and the two second cover plates 35 are arranged in a one-to-one correspondence with the two accommodating shells 34; and the second cover plates 35 are configured to close the open ends of the corresponding accommodating shells 34, respectively. A fixed connection between each second cover plate 35 and the corresponding accommodating shell 34 can be achieved by means of a snap-fit connection, a screw connection, or the like. In an embodiment, the driver member 6 is installed in the accommodating shell 34.

In other embodiments, the base unit 3 can also be configured as a telescopic structure, that is, the housing assembly 3a of the base unit 3 can include a multi-section telescopic mechanism, which can be adjusted according to the practical needs of the user.

As shown in FIG. 1, when the cervical traction device is normally used, the traction unit 1 is located above the base unit 3 in the vertical direction, that is, the traction unit 1 is arranged on a side of the base unit 3 close to the lower jaw of the user, such that the traction unit 1 can drive the lower jaw of the user to be lifted during rotation of the traction unit 1.

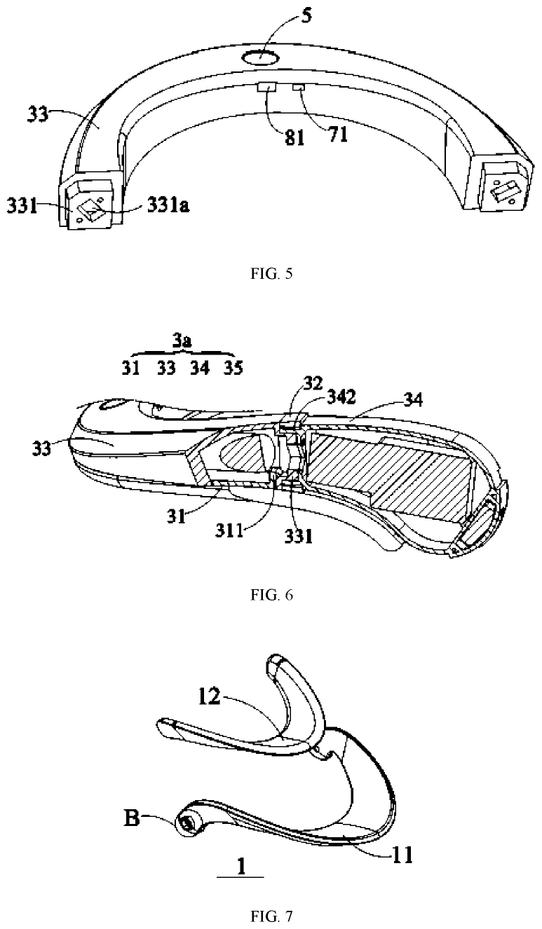

Referring to FIG. 7 again, the traction unit 1 includes: a support member 11 and a first flexible pad 12. The support member 11 is in a U-shaped structure. When the support member 11 is laid flat, the support member 11 gradually lifts up when extending from a middle of the support member 11 to the two ends thereof, that is, in the y-axis direction, the two ends of the support member 11 are located above the middle thereof, in this way, the middle of the support member 11 is adapted to a shape of the lower jaw of the human body, so that the support member 11 can fit the user's lower jaw and provide corresponding support. The first flexible pad 12 is disposed on a side of the support member 11 close to the lower jaw of the human body, and the first flexible pad 12 matches a shape of the middle of the support member 11. The first flexible pad 12 and the support member 11 may be in fixed connection by means of bonding, snap-fit connection, or the like.

In an embodiment, the first flexible pad 12 can be one selected from flexible pads made of, for example, a sponge, a silica gel, and a rubber. In an embodiment, the first flexible pad 12 is made of a sponge. It can be understood that a layer of cloth can be covered outside the sponge.

Two ends of the traction unit 1 are in connection with two ends of the base unit 3, respectively. Specifically, two ends of the support member 11 are in connection with the accommodating shells 34 at corresponding sides, respectively. The two ends of the support member 11 are connected with the accommodating shells 34 at corresponding sides in the same manner, which will be introduced hereinbelow alternatively.

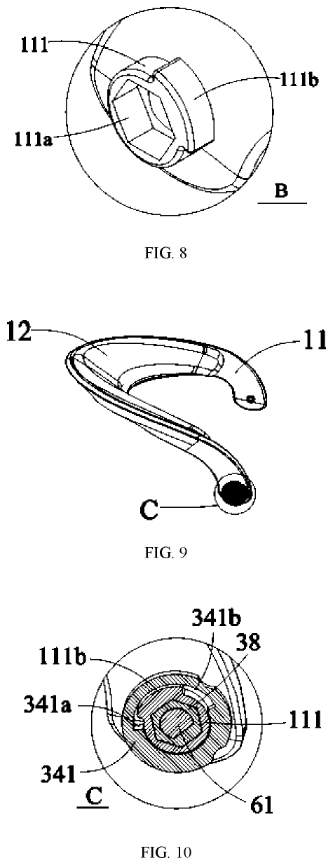

As shown in FIGS. 4, 7 to 10, a first connecting ring 111 is arranged at a side of the supporting member 11 close to a corresponding accommodating shell 34; a second connecting ring 341 is arranged at a side of the corresponding accommodating shell 34 close to the supporting member 11, and the first connecting ring 111 and the second connecting ring 341 are in plug-in connection.

An outer wall of the first connecting ring 111 is provided with a first limiting protrusion 111b. An inner wall of the second connecting ring 341 is provided with a second limiting protrusion 341a, and a travel groove 341b for the first limiting protrusion 111b is formed between two ends of the second limiting protrusion 341a. When the first connecting ring 111 and the second connecting ring 341 are in plug-in connection, the first connecting ring 111 is inserted into the second connecting ring 341, and the first limiting protrusion 111b is located in the travel groove 341b. When the traction unit 1 rotates, the first limiting protrusion 111b rotates in the travel groove 341b. When the traction unit 1 is rotated in place, the first limiting protrusion 111b abuts one end of the second limiting protrusion 341a. In this way, a rotation stroke of the first limiting protrusion 111b is limited, that is, the rotation stroke of the traction unit 1 is limited, so as to prevent the traction unit 1 from rotating excessively and causing injury to the user.

In normal wearing and use, the rotation stroke of the traction unit 1 limited by the travel groove 341b may correspond to an angle range of the traction unit 1 rotating from −10° below a horizontal plane to 30° above the horizontal plane. A maximum angle at which the traction unit 1 rotates above the horizontal plane can be configured to be 30° to avoid exceeding a bearing range of the human body. Thus, the angle at which the traction unit 1 drives the user's lower jaw to rotate upwards is adapted to the bearing range of the human body, and avoids injury to the human body during the traction process. The rotation angle of the traction unit 1 below the horizontal plane is not specifically limited, as long as it does not hinder the upward rotation of the traction unit 1.

In other embodiments, the angle at which the traction unit 1 rotates above the horizontal plane can also be configured to be other angles, such as 25°, 26°, and the like. The position and path length of the travel groove 341b are adaptively adjusted according to the rotation angle of the traction unit 1.

In an embodiment, the cervical traction device includes two driver members 6, the two driver members 6 are respectively disposed in the two accommodating shells 34, the two driver members 6 are connected with the two ends of the supporting member 11 in a one-to-one correspondence, and the two driver members 6 synchronously drive the support member 11 to rotate, that is, the two driver members 6 synchronously drive the traction unit 1 to rotate. The installation method of the two driver members 6 and the connection manners of the two driver members 6 with the supporting member 11 are the same, which are introduced hereinbelow in an alternative manner.

The driver member 6 is fixedly installed in the accommodating shell 34; an output shaft 61 of the driver member 6 extends out of the accommodating shell and is in connection with the first connecting ring 111 of the supporting member 11. Specifically, a cross section of the output shaft 61 is non-circular, and an end of the output shaft 61 close to the first connecting ring 111 is sleeved with a shaft sleeve 38, and a non-circular hole matching the output shaft 61 is provided inside the shaft sleeve 38, so as to enable the output shaft 61 to pass through. Thus, the output shaft 61 drives the shaft sleeve 38 to rotate synchronously. The outer cross section of the shaft sleeve 38 is also non-circular; correspondingly, a non-circular connection hole 111a is provided inside the first connecting ring 111, and the connection hole 111a matches the outer shape of the shaft sleeve 38; and the shaft sleeve 38 is embedded inside the first connecting ring 111. When the output shaft 61 drives the shaft sleeve 38 to rotate synchronously, the output shaft 61 drives the first connecting ring 111 to rotate synchronously, thereby driving the traction unit 1 to rotate.

In an embodiment, the end of the output shaft 61 inserted into the first connecting ring 111 can be limited in the first connecting ring 111 by means of screws and other structures. It can be understood that one end of the screw is in fixed connection with the output shaft 61, and the other end of the screw is limited to one side of the supporting member 11 is away from the accommodating shell 34, so as to avoid separation between the supporting member 11 and the accommodating shell 34.

In some other embodiments, the cervical traction device may include a driver member 6, and the driver member 6 is connected with one end of the traction unit 1, so that the driver member 6 drives the traction unit 1 to rotate.

As shown in FIG. 1, FIG. 12 and FIG. 13, the support unit 2 is arranged opposite to the traction unit 1, and during the cervical traction process, the support unit 2 is configured to provide support for the nape of the user. In the z-axis direction, the support unit 2 has an arc-shaped structure, and a middle part of the support unit 2 is recessed in a direction away from the traction unit 1. In the y-axis direction, the support unit 2 also has an arc-shaped structure, and the middle part of the support unit 2 is protruded towards the traction unit 1. In this way, the support unit 2 can be wrapped around the user's nape, to enable the support unit 2 to contact with the user's nape, thereby providing a larger area of support.

As shown in FIG. 12 and FIG. 13, the support unit 2 includes a support component 21 and a second flexible pad 22. The support component 21 forms a shape and structural basis of the support unit 2. In addition, the support component 21 enables the support unit 2 to enhance a support strength. The second flexible pad 22 is arranged on a side of the support component 21 close to the traction unit 1, that is, the second flexible pad 22 is disposed on the side of the support component 21 close to the user's nape, so as to improve the comfort degree of the user.

The second flexible pad 22 may be a flexible pad made of, for example, a sponge, a silica gel, and a rubber. In an embodiment, the second flexible pad 22 is made of a sponge. It can be understood that a layer of cloth can be covered outside the second flexible pad 22.

The support component 21 includes a first support plate 211 and a second support plate 212, and the first support plate 211 and the second support plate 212 are arranged to contact with each other. The first support plate 211 is provided with a sliding chute 211a and an installation groove 211b, and the installation groove 211b is in communication with the sliding chute 211a. In the z-axis direction, the sliding chute 211a penetrates through the first support plate 211. The installation groove 211b is arranged in a middle part of the first support plate 211, and the installation groove 211b extends along the x-axis direction.

In some other embodiments, the support unit 2 can also be configured to be a telescopic structure in the up-down direction. It can be understood that the support unit 2 includes an upper and a lower support structure, the lower support structure can support the user's nape, and the upper support structure can be configured to support the user's nape. The telescopic rod realizes the telescopic connection between the upper support structure and the lower support structure; which can therefore be adjusted by a user according to the practical need thereof, thereby providing a better use experience for the user.

In an embodiment, the support unit 2 is in connection with the base unit 3 through a connection unit 4. Specifically, the support unit 2 is installed on the connection unit 4; one end of the connection unit 4 is in hinge connection with one end of the base unit 3, and the other end of the connection unit 4 is in snap-fit connection with the other end of the base unit 3. Therefore, in use, the snap-fit connection between the connection unit 4 and the base unit 3 can be opened, and after the cervical traction device is worn on the user's neck, the connection unit 4 and the base unit 3 can be snap-fitted for connection, thereby realizing the fixation of the cervical traction device.

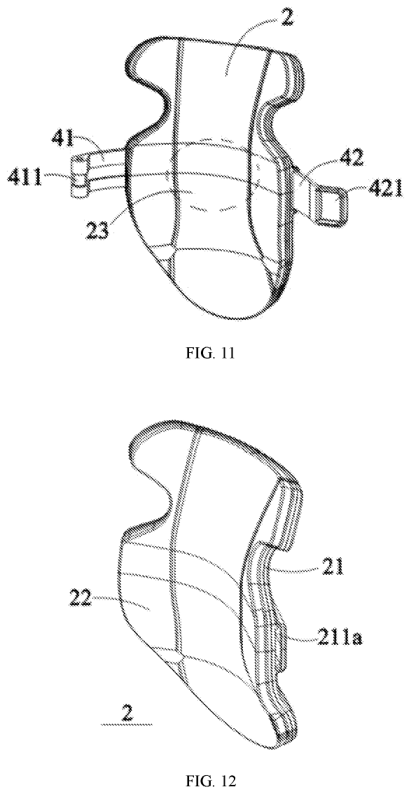

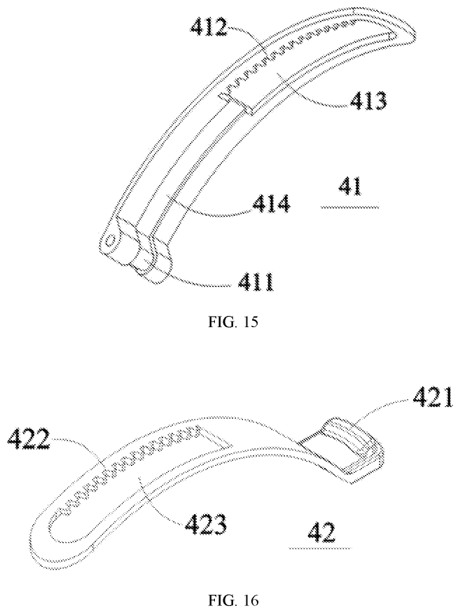

As shown in FIGS. 2, 3, 11, and 14-16, the connection unit 4 includes: a first connecting belt 41, a second connecting belt 42, and an adjustment knob 43. The adjustment knob 43 is rotatably installed in the installation groove 211b. One end of the first connecting belt 41 is hinged with the base unit 3, and the other end of the first connecting belt 41 is inserted into the sliding chute 211a and is engaged with the adjustment knob 43. One end of the second connecting belt 42 is in snap-fit connection with the base unit 3, and the other end of the second connecting belt 42 is inserted into the sliding chute 211a and is engaged with the adjustment knob 43.

Specifically, one end of the first connecting belt 41 is provided with a first connecting portion 411. One accommodating shell 34 of the base unit 3 is provided with a corresponding third connecting portion 36. The first connecting portion 411 and the third connecting portion 36 are rotatably connected by a rotating shaft (not shown in the figure), and the rotating shaft is extended along the y-axis direction. Therefore, the connection unit 4 can be rotated relative to one end of the base unit 3, that is, the support unit 2 is driven to rotate relative to the base unit 3.

A section of the first connecting belt 41 extending into the sliding chute 211a is provided with a first avoidance groove 413, a side wall of the first avoidance groove 413 is provided with a first rack portion 412, and the first rack portion 412 extends along a length direction of the first connecting belt 41.

One end of the adjustment knob 43 inserted into the installation slot 211b is in fixed connection with a gear 44. The gear 44 passes through the first avoidance groove 413, and the first rack portion 412 is engaged with the gear 44.

One end of the second connecting belt 42 is provided with a second connecting portion 421; correspondingly, the other accommodating shell 34 of the base unit 3 is provided with a fourth connecting portion 37. The second connecting portion 421 has a protruding block structure, and the fourth connecting portion 37 has a hole-like structure configured for engaging with the protruding block structure, so as to realize the snap-fit connection between the second connecting portion 421 and the fourth connecting portion 37. In use, when the cervical traction device is to be worn on the neck of the user, the second connection part 421 and the fourth connection part 37 can be opened, and the support unit 2 can be rotated to open the space between the support unit 2 and the traction unit 1 so as to receive the neck of the user. After the cervical traction device is put on, the second connecting part 421 is in snap-fit connection with the fourth connecting part 37. When the cervical traction device needs to be removed, the user only needs to press the second connecting portion 421 to pop out the second connecting portion 421 from the fourth connecting portion 37 to open the space between the support unit 2 and the traction unit 1. Therefore, it is convenient for the user to wear or remove the cervical traction device, and the operation of the user is convenient.

A section of the second connecting belt 42 extending into the chute 211a is provided with a second avoidance groove 423, and a second rack portion 422 is disposed at one side wall of the second avoidance groove 423; the second connecting belt 42 contact with the first connecting belt 41. One end of the gear 44 away from the adjustment knob 43 extends into the second escape groove 423 and is engaged with the second rack portion 422.

In an embodiment, in a normal use state, in the y-axis direction, the first rack portion 412 and the second rack portion 422 are respectively disposed on the upper and lower sides of the gear 44. Therefore, by driving the gear 44 to rotate by the adjustment knob 43, the first connecting belt 41 and the second connecting belt 42 can move toward or away from each other, that is, the first connecting portion 411 and the second connecting portion 421 are driven to approach to each other or move away from each other, thereby adjusting the length of the connection unit 4 and adapting to the needs of users of different body shapes. In use, the user can adjust the length of the connection unit 4 as required, so that the support unit 2 can contact with the nape.

In an embodiment, the support unit 2 is further provided with a heating sheet (not shown in the figure), and the heating sheet is configured to perform hyperthermia on the neck of the user. Specifically, the heating sheet is disposed on a side of the support component 21 close to the second flexible pad 22; and the heating sheet is disposed corresponding to a contact position between the second flexible pad 22 and the neck.

The heating sheet can be provided with multiple heating gear positions, which can be adjusted by the user according to practical needs. Exemplarily, the heating sheet can be provided with three gear positions. At a first gear position, the temperature of the heating sheet can be controlled at 40° C.; at a second gear position, the temperature of the heating sheet can be controlled at 45° C.; and at a third gear position, the temperature of the heating sheet can be controlled at 50° C. The can choose and adjust the heating gear according to practical needs.

In some other embodiments, the support unit 2 can also be provided with a magnetic therapy sheet, which can provide magnetic therapy for the user. The magnetic therapy sheet can be arranged at a side of the support component 21 close to the second flexible pad 22; and the magnetic therapy sheet is disposed corresponds to a contact position between the second flexible pad 22 and the nap.

It can be understood that, as shown in FIG. 3, the cervical traction device is further provided with a main control board 8, and the main control board 8 is fixedly arranged in the housing assembly 3a of the base unit 3. Both the heating sheet and the driver member 6 are in electric connection with the main control board 8, and the main control board 8 is configured to control the action of the cervical traction device.

Among them, the main control board 8 can control the adjustment of the gear position of the heating sheet. The main control board 8 can control the action stroke of the driver member 6, thereby preventing the driver member 6 from driving the traction unit 1 to rotate excessively, and further ensuring the safety of the cervical traction device. The driving part 6 may adopt a motor having a self-locking function, and the self-locking can be triggered when the action is in place. The main control board 8 can be provided with a controller and a memory correspondingly to adjust and control the work of the cervical traction device.

As shown in FIG. 14, a side of the first connecting strip 41 defines therein a wire groove 414, and the wire groove 414 extends to a position of the first connecting portion 411. Therefore, it is convenient for the wires led out from the base unit 3 to be introduced into the support unit 2 along the wire groove 414 and to be in electric connection with the heating sheet. Moreover, the problem of stranded wires when the connection unit 4 is stretched or rotated can be avoided.

Optionally, the main control board 8 is further provided with an emergency stop button 5. In use, during the traction process, if the user feels uncomfortable, the user can directly stop the action of the cervical traction device via the emergency stop button 5.

A power source 7 is also arranged in the housing assembly 3a, and the power source 7 is in electric connection with the main control board 8, so as to provide electric energy for the operation of the cervical traction device.

In an embodiment, the power source 7 may adopt a rechargeable battery. Correspondingly, the main control board 8 is provided with a charging interface 81 to realize the charging of the power source 7. In an embodiment, the main control board 8 may further be provided with an indicator group 71 for displaying a power level of the power supply 7. The indicator group 71 is disposed close to the housing assembly 3a. Correspondingly, the housing assembly 3a is provided with a light-transmitting portion, so that the light transmitted from the indicator group 71 can penetrate outwards and is visible to the user. Optionally, the indicator group 71 may include LED lights having two colors, for example, yellow and green. When the indicator group 71 displays yellow, it is indicated that the user needs to charge the power source; and when the indicator group 71 displays green, it is indicated that the power supply 7 is fully charged.

In other embodiments, the indicator group 71 may also include LED lights having three colors, for example, red, yellow, and green. When the indicator group 71 displays yellow, it is indicated that the user needs to charge the power source; when the indicator group 71 displays red, it is indicated that the power of the power supply 7 is extremely low, and the user needs to charge the power source as soon as possible; and when the indicator group 71 displays green, it is indicated that the power supply 7 is sufficient.

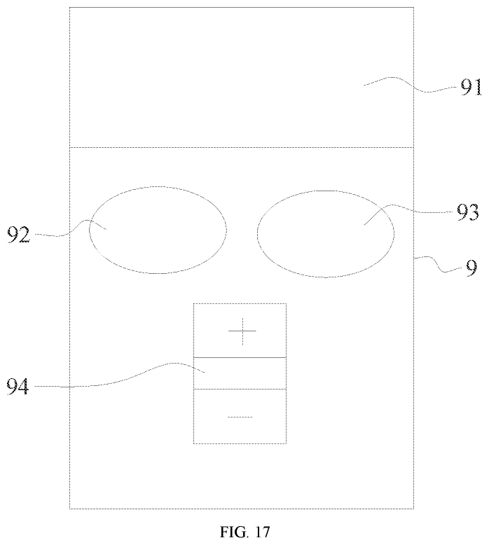

As shown in FIG. 17, in an embodiment, the cervical traction device is further equipped with a remote controller 9, and both the remote controller 9 and the main control board 8 are provided with corresponding communication units to achieve the communication between the remote controller 9 and the main control board 8. Therefore, the operation of the cervical traction device can be controlled by the remote controller 9.

In some embodiments, the communication unit may include one or more wireless communication manners, including Bluetooth and Wifi.

In an embodiment, the remote controller 9 is provided with a display area 91, a temperature key 92, an emergency stop key 93, and a traction key 94. The display area 91 can be configured to display the working parameters of the cervical traction device, such as the heating temperature of the heating sheet. The temperature key 92 can be configured to adjust the gear position of the heating sheet. The traction key 94 can be operated by the user to control the traction action of the cervical traction device. For example, when the user presses the key “+”, the cervical traction device is activated, and the driver member 6 drives the traction unit 1 to rotate in a direction towards the support unit 2; when the user presses the key “−”, the driver member 6 is controlled by the main control board 8 to work, and drives the traction unit 1 to rotate and reset in the direction away from the support unit 2.

In some embodiments, the user can also fine-tune the rotation angle of the traction unit 1 through the traction key 94. Exemplarily, the user can double-click the key “+” to start the driver member 6 to drive the traction unit 1 to perform the traction action, and the user can click the key “+” to control the driver member 6 to drive the traction unit 1 to fine-tune a relatively small angle towards the support unit 2, to meet the user's traction needs. Similarly, the user can start the driver 6 to drive the traction unit 1 to reset by double-clicking the key “−”, and the user can control the driver member 6 to drive the traction unit 1 to fine-tune a relatively small angle away from the support unit 2 by clicking the key “−”, to meet the user's traction needs, improve the comfort degree of the user, and avoid harm to the user. The fine-tuning angle can be set to a small angle such as ±0.5°, ±1°, and the like, and the fine-tuning angle can be set during the production process of the cervical traction device, so as to prevent users from setting an unreasonable rotation angle by themselves. It can be understood that the angle at which the driver member 6 drives the traction unit 1 to rotate can also be set during the production process of the cervical traction device, so as to prevent the user from setting an unreasonable rotation angle and causing harm to the human body.

In an embodiment, the cervical traction device further includes a pressure detection assembly (not shown in the figure) for detecting a traction force applied by the traction unit 1 to the user. The pressure detection assembly is in electric connection with the main control board 8. The pressure value detected by the pressure detection assembly can be displayed on the display area of the remote controller 9, which can be visible to the user.

The pressure detection assembly is arranged at least one of the support unit 2 and the traction unit 1. It can be understood that the pressure detection assembly is located at a position where the support unit 2 contacts the back of the head and/or is located at a position where the traction unit 1 contacts the lower jaw, which enables the pressure detection assembly accurately to sense the magnitude of the traction force. In this embodiment, the pressure detection assembly is arranged at the support unit 2, and the contact position between the support unit 2 and the back of the head is defined as a force receiving area 23.

In some specific embodiments, the pressure detection assembly includes at least four pressure sensors, and the pressure sensors are evenly distributed in the force bearing area 23 of the support unit 2, so that the pressure detection assembly can accurately detect the traction force exerted by the traction unit 1 on the user. For example, the pressure detection assembly may include four, five, six, eight, nine, ten, and other numbers of pressure sensors.

It can be understood that, in some other embodiments, the pressure detection assembly may further include one, two, three, and other numbers of pressure sensors. The pressure sensors are distributed unevenly in the force bearing area 23 of the support unit 2.

When the pressure detection assembly detects that the traction force reaches the preset traction force, the main control board 8 can control the driver member 6 to stop the action, so as to avoid excessive traction of the traction unit 1, which would otherwise cause injury to the user.

The preset traction force can be set according to the user's tolerance ability, and the preset traction force can be converted into a corresponding preset traction weight. The preset traction weight can be set to be between 1/12 and ⅕ of the user's body weight. Exemplarily, for a user having a weight of about 50 kg, the traction weight that the user can tolerate is between 4.25 kg and 10 kg. Therefore, the preset traction force of the cervical traction device can be set at between 41.65 N and 98 N, and a maximum preset traction force can be set at 98 N. In an embodiment, the preset traction weight can be set to a maximum of 20 kg, and the corresponding preset traction force can be set to a maximum of 196 N, so as to avoid exceeding the human body's tolerance limit. It can be understood that the user's weight mentioned in this application is a healthy weight. The preset traction force can be set according to the user's needs in the production of the cervical traction device, and the user does not need to adjust it by himself/herself.

In use, the user wears the cervical traction device around the neck, so that the support unit 2 corresponds to the nape, and the traction unit 1 corresponds to the lower jaw. The driver member 6 can be controlled to start through the key “+” on the remote controller 9, and the driver member 6 drives the traction unit 1 to gradually rotate towards the support unit 2. During this period, the traction unit 1 gradually contacts the user's lower jaw, and drives the lower jaw to gradually lift. When the traction unit 1 rotates to a preset angle, the main control panel 8 controls the driver member 6 to stop moving, so that the user's neck is maintained in the traction position. Then, the user can control the driver member 6 to drive the traction unit 1 to rotate and reset in a reverse direction through the key “−” on the remote controller 9 to release the user's lower jaw.

It can be understood that, in some embodiments, a retention time of the traction unit 1 at the traction position can be preset in the main control panel 8, and after the retention time reaches a preset time, the main control panel 8 controls the driver member 6 to automatically rotate in the reverse direction, so that the traction unit 1 can rotate reversely to reset and to release the user's jaw. The preset time can be set according to the general user's human condition, and can also be customized according to the needs of the user. Exemplarily, the preset time can be set to a time length such as 25s, 30s, or the like. In use, the user can perform multiple traction actions through the cervical traction device to achieve a better traction effect.

To sum up, when the cervical traction device provided by the present application performs cervical traction on the user, the mechanical limit at the connection between the traction unit 1 and the base unit 3, the self-locking function of the driver member 6, and the detection of the pressure detection assembly can be adopted to prevent the traction unit 1 from exceeding the preset position, which can effectively avoid excessive traction of the cervical traction device exerted on the user when providing cervical traction to the user, thereby avoiding injury to the user, and ensuring the user's safety. The cervical traction device provided by the present application features in intelligence, light weight, and portability, and is convenient for patients to use at home or outdoors. Meanwhile, the cervical traction device provided in the present application can be applied to the cervical spondylosis patients of the following types:
1. Patients having cervical soreness, straightening cervical curvature, and cervical kyphosis caused by long-term bowing and working at the desk;
2. Patients having neck pain, arm numbness, and nerve root compression;
3. Patients having partial stiff limbs, faltering walking, and spinal cord compression;
4. Patients having cricoaxial subluxation (that is, stiff neck);
5. Patients in need of immobilization after cervical spondylosis surgery.

The above are only optional embodiments of the present application, which are not intended to limit the present application. Various modifications and variations of the present application are possible for those skilled in the art. Any modification, equivalent replacement, improvement, etc. made within the spirit and principle of the present application shall be included within the scope of the claims of the present application.

1. A cervical traction device, comprising:
a base unit, a traction unit, a support unit, and a driver member;
wherein
the traction unit is rotatably installed at the base unit, and the traction unit is configured to support a lower jaw of a user;
the support unit is in connection with the base unit and is arranged opposite to the traction unit; the support unit is configured to support a nape of the user; and
the driver member is configured to drive the traction unit to rotate, to make the traction unit turn to or away from the support unit.

2. The cervical traction device according to claim 1, wherein
the driver member is one of two driver members, and the two driver members are disposed at two ends of the base unit, respectively; and
the two driver members are respectively connected with two ends of the traction unit in a one-to-one correspondence, and the two driver members are configured to synchronously drive the traction unit to rotate.

3. The cervical traction device according to claim 1, wherein
the traction unit is provided with a first connecting ring configured to connect with the base unit, and an outer wall of the first connecting ring is provided with a first limiting protrusion;
the base unit is provided with a corresponding second connecting ring, and an inner wall of the second connecting ring is provided with a second limiting protrusion; and
the first connecting ring is inserted into the second connecting ring, and the second limiting protrusion is configured to limit a rotation stroke of the first limiting protrusion and in turn to limit a rotation stroke of the traction unit.

4. The cervical traction device according to claim 1, wherein at least one of the traction unit and the support unit is provided with a pressure detection assembly; and the pressure detection assembly is configured to detect a magnitude of a traction force that the traction unit exerts on the user.

5. The cervical traction device according to claim 4, wherein the pressure detection assembly comprises at least one pressure sensor.

6. The cervical traction device according to claim 1, wherein the support unit is provided thereon with a magnetic therapy sheet; and the magnetic therapy sheet is arranged at a side of the support unit that is close to a nape of the user.

7. The cervical traction device according to claim 1, wherein the support unit is further provided thereon with a heating sheet; and the heating sheet is arranged at a side of the support unit that is close to a nape of the user.

8. The cervical traction device according to claim 1, wherein a first flexible pad is arranged at a side of the traction unit close to the user, a second flexible pad is arranged at a side of the support unit close to the user, and a third flexible pad is arranged at a side of the base unit close to the user.

9. The cervical traction device according to claim 1, wherein the support unit is in connection with the base unit through a connection unit, and the support unit is installed at the connection unit; and one end of the connection unit is in hinge connection with the base unit, and another end of the connection unit is in snap-fit connection with the base unit.

10. The cervical traction device according to claim 9, wherein
the connection unit comprises: a first connecting belt, a second connecting belt, and an adjustment knob; the adjustment knob is rotatably installed at the support unit;
one end of the first connecting belt is in hinge connection with the base unit, and another end of the first connecting belt is inserted into the support unit and engaged with the adjustment knob;
one end of the second connecting belt is in snap-fit connection with the base unit, and another end of the second connecting belt is inserted into the support unit and engaged with the adjustment knob; and
the adjustment knob is configured to drive the first connecting belt and the second connecting belt to move toward or away from each other by rotation, so as to lengthen or shorten a length of the connection unit.

11. The cervical traction device according to claim 8, wherein the traction unit further comprises a support member; the first flexible pad is arranged at a side of the support member close to the lower jaw of the human body user; and the first flexible pad matches a shape of a middle portion of the support member.

12. The cervical traction device according to claim 8, wherein the support unit further comprises a support component in connection with the base unit, and the second flexible pad is disposed at a side of the support component close to the traction unit.

13. The cervical traction device according to claim 12, wherein the support component comprises a first support plate and a second support plate, the first support plate and the second support plate are attached to each other, the first support plate is provided therein with a sliding chute and an installation groove, and the installation groove is in communication with the sliding chute.

14. The cervical traction device according to claim 8, wherein the base unit further comprises a housing assembly, and the third flexible pad is in connection with the housing assembly.
